(19) United States
(12) Patent Application Publication
(10) Pub. No.: US 2023/0248986 A1
(43) Pub. Date: Aug. 10, 2023

(54) DEFIBRILLATOR

(71) Applicant: Nihon Kohden Corporation, Tokyo (JP)

(72) Inventors: Yuji Yamada, Saitama (JP); Ryosuke Kuno, Saitama (JP); Fumihito Iwai, Saitama (JP)

(73) Assignee: Nihon Kohden Corporation, Tokyo (JP)

(21) Appl. No.: 18/158,840

(22) Filed: Jan. 24, 2023

(30) Foreign Application Priority Data
Feb. 4, 2022 (JP) 2022-016386

(51) Int. Cl. A61N 1/39

(52) U.S. Cl. CPC A61N 1/3975

(57) ABSTRACT

A defibrillator includes: processing circuitry; and a memory storing a program. When executed on the processing circuitry, the program causes the processing circuitry to: calculate battery usage of a battery configured to supply power to the defibrillator; record, on a battery memory, information related to the battery usage; and record, on the battery memory, battery information indicating battery failure in response to voltage of the power supplied from the battery being below a threshold.

### CROSS-REFERENCE TO RELATED APPLICATION

This application is based on and claims priority under 35 USC § 119 from Japanese Patent Application No. 2022-016386, filed on Feb. 4, 2022, the entire contents of which are incorporated herein by reference.

### TECHNICAL FIELD

The present disclosure relates to a defibrillator.

### BACKGROUND

Defibrillators have been rapidly spread recently. A defibrillator is configured to give a patient back the heart function by giving a strong electric shock to the heart having stopped beating suddenly due to ventricular fibrillation. Some defibrillators have a self-diagnosis function to self-diagnose whether there is failure or not regularly. For example, JP2016-500286A discloses a defibrillator configured to conduct self-diagnosis regularly at a first frequency or a second frequency, and JP2006-043270A discloses a defibrillator configured to conduct self-diagnosis to detect battery failure.

The defibrillator of JP2016-500286A can detect failure while holding down power consumption by dynamically controlling the frequency of the self-diagnosis. The defibrillator of JP2006-043270A is configured to notify an administrator of battery failure if it is detected during the self-diagnosis.

Suppose that first self-diagnosis, during which current supplied from a battery is low, is conducted and that second self-diagnosis, during which current supplied from the battery is high, is conducted less frequently than the first self-diagnosis. In this case, even if a battery failure in which internal impedance is too high is detected during a second self-diagnosis, there is a possibility that the following first self-diagnosis cannot detect the battery failure since a voltage drop is small as well as the current supplied from the battery during first self-diagnosis. Therefore, there is room for improvement in terms of recognizability of battery failure.

An object of the present disclosure is to provide a defibrillator that enables users or administrators to recognize battery failure easily even if current supplied from a battery is low during self-diagnosis.

### SUMMARY

A defibrillator according to an aspect of the present disclosure includes:

processing circuitry; and

a memory storing a program, in which,

when executed on the processing circuitry, the program causes the processing circuitry to:
calculate battery usage of a battery configured to supply power to the defibrillator;
record, on a battery memory, information related to the battery usage; and
record, on the battery memory, battery information indicating battery failure in response to voltage of the power supplied from the battery being below a threshold.

The present disclosure can provide a defibrillator that enables users or administrators to recognize battery failure easily even if current supplied from a battery is low during self-diagnosis.

### BRIEF DESCRIPTION OF DRAWINGS

Exemplary embodiments of the present disclosure will be described in detail based on the following figures, wherein:

FIG. 1 illustrates an exterior of a defibrillator according to an embodiment of the present disclosure; and

FIG. 2 is a block diagram of the defibrillator.

### DESCRIPTION OF EMBODIMENT

In the following, an embodiment of the present disclosure will be described with reference to the drawings. Descriptions of components having the same reference numerals as those of components already described will be omitted for the sake of simplicity. Dimensions of components shown in the drawings may be different from the actual dimensions for the sake of convenience.

Configuration of Defibrillator

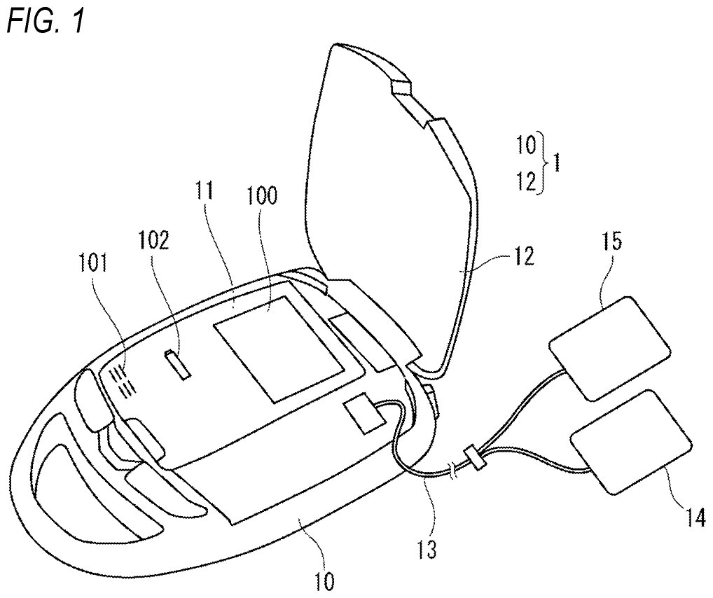

FIG. 1 illustrates an exterior of a defibrillator 1 according to an embodiment of the present disclosure. The defibrillator 1 can include a body 10 (housing body) and a lid 12. The defibrillator 1 is connectable to a battery pack 11, a cable 13, and electrode pads 14 and 15.

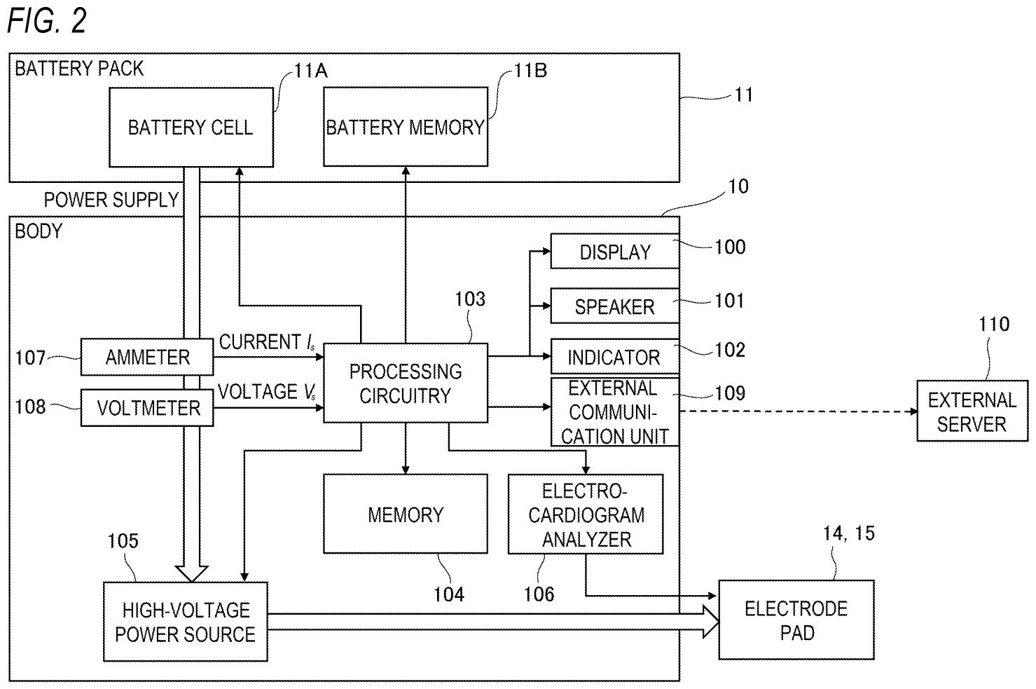

The battery pack 11 can include a battery cell 11A (see FIG. 2) and is configured to supply power for operating the body 10 and the electrode pads 14 and 15 to the body 10. The battery pack 11 is connectable to the base of the body 10. The lid 12 is configured to cover the body 10.

In response to a power button on the body 10 being pushed, the main power source of the defibrillator 1 is turned on. The defibrillator 1 may be an automated external defibrillator (AED) whose main power source is on when the lid 12 is open and off when the lid 12 is closed.

The cable 13 is configured to connect the electrode pads 14 and 15 to the body 10 electrically. The electrode pads 14 and 15 are attachable to a subject to enable electrocardiogram analysis and/or treatment using an electric shock for the subject.

The body 10 can include a display 100, a speaker 101, and an indicator 102 as user interfaces.

The display 100 is configured to display graphics and/or characters to visually provide rescuers with information on, for example, how to attach the electrode pads 14 and 15, how to administer an electric shock, and whether the state of the defibrillator 1 is normal or abnormal.

The speaker 101 is configured to reproduce sounds to aurally provides the rescuers with information on, for example, how to attach the electrode pads 14 and 15, how to administer the electric shock, warning that the body 10 is being charged with a high voltage, timing of the electric shock, and whether the state of the defibrillator 1 is normal or abnormal.

The indicator 102 can includes a light configured to be turned on or to blink to visually provide the rescuers with information on, for example, whether the state of the defibrillator 1 is normal or abnormal.

FIG. 2 is a block diagram of the defibrillator 1. The battery pack 11 can include the battery cell 11A and a battery memory 11B. The body 10 can include the display 100, the speaker 101, the indicator 102, processing circuitry 103 (for example, one or more processors), a memory 104, a high-voltage power source 105, an electrocardiogram analyzer 106, an ammeter 107, a voltmeter 108, and an external communication unit 109.

In response to the power button being pushed and the main power source of the defibrillator 1 being turned on, the processing circuitry 103 loads programs and data stored in the memory 104 and runs the programs to control the defibrillator 1.

The processing circuitry 103 loads graphic data that is stored in advance in the memory 104 and is related to the information on, for example, how to attach the electrode pads 14 and 15, how to administer the electric shock, and whether the state of the defibrillator 1 is normal or abnormal, and transmits the graphic data to the display 100. The display 100 receives and displays the graphic data.

The processing circuitry 103 transmits control signals to the indicator 102 to control the light of the indicator in order to notify the rescuers whether the state of the defibrillator 1 is normal or abnormal. The indicator 102 receives the control signals, and the light of the indicator 102 is turned on or blinks based on the control signals.

The processing circuitry 103 loads sound data that is stored in advance in the memory 104 and is related to the information on, for example, how to attach the electrode pads 14 and 15, how to administer the electric shock, warning that the body 10 is being charged with a high voltage, timing of the electric shock, and whether the state of the defibrillator 1 is normal or abnormal, and transmits the sound data to the speaker 101. The speaker 101 receives the sound data and reproduces sounds based on the sound data.

The processing circuitry 103 controls the battery cell 11A and an internal capacitor of the high-voltage power source 105 such that the internal capacitor is charged by the battery cell 11A or is discharged into the electrode pads 14 and 15. The ammeter 107 and the voltmeter 108 is configured to measure current Iₛ and voltage Vₛ of power supplied from the battery cell 11A to the body 10.

The processing circuitry 103 determines whether the electrode pads 14 and 15 are attached to the subject and controls the electrode pads 14 and 15 and the electrocardiogram analyzer 106 to cause the electrocardiogram analyzer 106 to conduct electrocardiogram analysis.

The memory 104 stores programs and data for controlling the defibrillator 1. The electrocardiogram analyzer 106 conducts the electrocardiogram analysis on the subject using the electrode pads 14 and 15.

The processing circuitry 103 conducts self-diagnosis to diagnose whether the defibrillator 1 can operate normally, or whether the state of the defibrillator 1 is normal or abnormal. The processing circuitry 103 records results of the self-diagnosis on the memory 104 and transmits the results to an external server (management server) 110 via the external communication unit 109.

Self-Diagnosis

The self-diagnosis will be described in detail. The processing circuitry 103 is configured to conduct at least two types of self-diagnosis: one is first self-diagnosis, and another is second self-diagnosis.

In the first and the second self-diagnosis, at least remaining battery level and battery voltage of the battery cell 11A are checked. Based on results of the check of the remaining battery level and the battery voltage, it is comprehensively determined whether the state of the defibrillator is normal or abnormal.

Check of Remaining Battery Level

First, the processing circuitry 103 checks the remaining battery level at the start of the self-diagnosis. Specifically, the processing circuitry 103 loads the remaining battery level Iᵣ and full battery level I_{f} of the battery cell 11A that are stored in the battery memory 11B. The full battery level I_{f} is, for example, 2000 mAh.

Next, the processing circuitry 103 calculates a remaining battery level ratio Iᵣᵣ of the remaining battery level Iᵣ to the full battery level I_{f} and compares the remaining battery level ratio Iᵣᵣ with a predetermined threshold Iₜₕ. The predetermined threshold Iₜₕ is, for example, 5%. I_{f} the remaining battery level ratio Iᵣᵣ exceeds the predetermined threshold Iₜₕ, the processing circuitry 103 determines that the remaining battery level Iᵣ is normal. I_{f} the remaining battery level ratio Iᵣᵣ is below the predetermined threshold Iₜₕ, the processing circuitry 103 determines that the remaining battery level Iᵣ is abnormal. The processing circuitry 103 records results of the determination as to whether the remaining battery level Iᵣ is normal or abnormal on the memory 104.

In addition, the processing circuitry 103 checks the remaining battery level at the end of the self-diagnosis. Specifically, the processing circuitry 103 measures the current Iₛ supplied from the battery cell 11A to the body 10. The processing circuitry 103 calculates a fluctuation ΔIᵤ in battery usage, based on the time integral of the current Iₛ. The processing circuitry 103 calculates the remaining battery level Iᵣ at the end of the self-diagnosis by subtracting the fluctuation ΔIᵤ in the battery usage from the remaining battery level Iᵣ at the start of the self-diagnosis. The processing circuitry 103 calculate the remaining battery level ratio Iᵣᵣ at the end of the self-diagnosis of the remaining battery level Iᵣ to the full battery level I_{f} and compares the remaining battery level ratio Iᵣᵣ at the end of the self-diagnosis with the predetermined threshold Iₜₕ. I_{f} the remaining battery level ratio Iᵣᵣ exceeds the predetermined threshold Iₜₕ, the processing circuitry 103 determines that the remaining battery level Iᵣ is normal. I_{f} the remaining battery level ratio Iᵣᵣ is below the predetermined threshold Iₜₕ, the processing circuitry 103 determines that the remaining battery level Iᵣ is abnormal. The processing circuitry 103 records the remaining battery level Iᵣ at the end of the self-diagnosis on the battery memory 11B and records results of the determination as to whether the remaining battery level Iᵣ is normal or abnormal on the memory 104.

Instead of the remaining battery level Iᵣ and the remaining battery level ratio Iᵣᵣ, battery usage Iᵤ and a battery usage ratio Iᵤᵣ may be checked during the check of the remaining battery level, and the processing circuitry 103 may record both the remaining battery level ratio Iᵣᵣ and the battery usage ratio Iᵤᵣ on the battery memory 11B. In addition, the memory 104 may store identification information for a plurality of batteries and the full battery level I_{f} of each battery in association with each other, and the battery memory 11B may store the identification information for one of the batteries. In this case, the processing circuitry 103 may load the remaining battery level Iᵣ of and the identification information for a battery from the battery memory 11B and acquire, from the memory 104, the full battery level I_{f} of the battery, based on the identification information for the battery.

By checking the remaining battery level in this manner, it becomes possible to detect a battery failure in which the battery cell 11A does not have enough power for operating the defibrillator 1.

Check of Battery Voltage

The processing circuitry 103 checks the battery voltage by comparing the voltage Vₛ with a predetermined threshold Vₜₕ. I_{f} the voltage Vₛ exceeds the predetermined threshold Vₜₕ, the processing circuitry 103 determines that the battery voltage is normal. I_{f} the voltage Vₛ is below the predetermined threshold Vₜₕ, the processing circuitry 103 determines that the battery voltage is abnormal. The processing circuitry 103 records results of the determination as to whether the battery voltage is normal or abnormal on the memory 104.

By checking the battery voltage in this manner, it becomes possible to detect a battery failure in which internal impedance of the battery cell 11A is too high.

Determination of State of Defibrillator

The processing circuitry 103 loads the results of the determination, which are recorded on the memory 104, as to whether the remaining battery level Iᵣ is normal or abnormal and whether the battery voltage is normal or abnormal and comprehensively determines whether the state of the defibrillator 1 is normal or abnormal based on at least the determination results. If it is determined that the remaining battery level Iᵣ and the battery voltage are normal, the processing circuitry 103 may determine that the state of the defibrillator 1 is normal. If it is determined that the remaining battery level Iᵣ and/or the battery voltage is abnormal, the processing circuitry 103 may determine that the state of the defibrillator 1 is abnormal. The processing circuitry 103 records results of the determination as to whether the defibrillator is normal or abnormal on the memory 104.

The processing circuitry 103 causes the display 100, the speaker, and/or the indicator 102 to output the results of the determination as to whether the state of the defibrillator 1 is normal or abnormal and transmits the results to the external server 110 via the external communication unit 109.

By determining the state of the defibrillator 1 in this manner, it becomes possible for users or administrators of the defibrillator 1 to recognize whether the state of the defibrillator 1 is normal or abnormal.

First and Second Self-Diagnosis

In the first self-diagnosis, at least the remaining battery level Iᵣ and the battery voltage of the battery cell 11A are checked regularly, for example, once a day. In the first self-diagnosis, when the remaining battery level Iᵣ is checked, the current Iₛ is measured with the processing circuitry 103 causing first current to be supplied from the battery cell 11A to the body 10.

In the second self-diagnosis, at least the remaining battery level Iᵣ and the battery voltage of the battery cell 11A are checked regularly at intervals longer than that for the first self-diagnosis, for example, once a month. In the second self-diagnosis, when the remaining battery level Iᵣ is checked, the current Iₛ is measured with the processing circuitry 103 causing second current, which is higher than the first current, to be supplied from the battery cell 11A to the body 10.

The first and the second self-diagnosis differ each other in the current (first and second current) supplied from the battery cell 11A to the body 10 and the intervals.

In the second self-diagnosis, the high-voltage power source 105 is charged by the battery cell 11A to diagnose whether the high-voltage power source 105 can be charged normally. On the other hand, in the first self-diagnosis, the high-voltage power source 105 is not charged by the battery cell 11A. Therefore, the second current is higher than the first current, and the second self-diagnosis consumes much power of the battery cell 11A. The second self-diagnosis cannot be conducted frequently, and the intervals for the second self-diagnosis are longer than those for the first self-diagnosis.

### EMBODIMENT

Table 1 shows results of the self-diagnosis for the defibrillator 1 according to the present embodiment when the battery cell 11A is normal (when the internal impedance is small).

TABLE 1

| Number of Self-diagnosis | | 1 | 2 | 3 | 4 | 5 |
|---|---|---|---|---|---|---|
| Type of Self-diagnosis | | First | | Second | First | |
| Internal Impedance of Battery | | Normal | | | | |
| Cell | | | | | | |
| High-voltage Power Source | | No | | Yes | No | |
| Is Charged? | | | | | | |
| Voltage Drop | | Small | | | | |
| Check of | Battery Usage | 1% | 2% | 5% | 6% | 6% |
| Remaining | Ratio Iᵤᵣ | | | | | |
| Battery Level | Remaining | 99% | 98% | 95% | 94% | 94% |
| Iᵣ | Battery | | | | | |
| | Level Ratio Iᵣᵣ | | | | | |

| | | |
|---|---|---|
| | Results | Normal |
| Check of | Results | Normal |
| Battery Voltage | | |
| Comprehensive | Results | Normal |
| Determination | | |

As shown in Table 1, if the first self-diagnosis is conducted when the battery cell 11A is normal, the first current, which is low, is supplied from the battery cell 11A to the body 10 without charging the high-voltage power source 105 by the battery cell 11A. Therefore, the battery usage ratio Iᵤᵣ and the remaining battery level ratio Iᵣᵣ (hereinafter, the battery usage ratio Iᵤᵣ and the remaining battery level ratio Iᵣᵣ are generically referred to as “battery information”) are normal value. In addition, since the first current and the voltage drop of the voltage Vₛ measured by the voltmeter 108 are small, the voltage Vₛ exceeds the predetermined threshold Vₜₕ. Therefore, it is determined that the battery voltage is normal and that the state of the defibrillator 1 is normal.

Based on the results of the self-diagnosis, information indicating that the state of the defibrillator 1 is normal is recorded on the memory 104, and battery information indicating that the battery cell 11A is normal (specifically, according to Table 1, the battery usage ratio Iᵤᵣ is 1-6%, and the remaining battery level ratio Iᵣᵣ is 94-99%) is recorded on the battery memory 11B.

If the second self-diagnosis is conducted when the battery cell 11A is normal, the high-voltage power source 105 is charged by the battery cell 11A, and the second current, which is high, is supplied from the battery cell 11A to the body 10. However, since the intervals for the second self-diagnosis are long (the frequency is low), the fluctuation ΔIᵤ in the battery usage is small, and the battery usage ratio Iᵤᵣ and the remaining battery level ratio Iᵣᵣ are normal value. In addition, since the battery cell 11A is normal and has small internal impedance, the voltage drop of the voltage Vₛ measured by the voltmeter 108 is small, and the voltage Vₛ exceeds the predetermined threshold Vₜₕ. Therefore, it is determined that the battery voltage is normal and that the state of the defibrillator 1 is normal.

Based on the results of the self-diagnosis, information indicating that the state of the defibrillator 1 is normal is recorded on the memory 104, and battery information indicating that the battery cell 11A is normal (specifically, according to Table 1, the battery usage ratio Iᵤᵣ is 5%, and the remaining battery level ratio Iᵣᵣ is 95%) is recorded on the battery memory 11B.

In order to explain results of the self-diagnosis for the defibrillator 1 according to the present embodiment when the battery cell 11A is abnormal (when the internal impedance is large), first, results of self-diagnosis for conventional defibrillator when its battery cell is abnormal (when the internal impedance is large) are shown in Table 2 for comparison.

TABLE 2

| Number of Self-diagnosis | | 1 | 2 | 3 | 4 | 5 |
|---|---|---|---|---|---|---|
| Type of Self-diagnosis | | First | | Second | First | |
| Internal Impedance of Battery | | Normal | Ab- | | | |
| Cell | | | normal | | | |
| High-voltage Power Source Is | | No | | Yes | No | |
| Charged? | | | | | | |
| Voltage Drop | | Small | | Large | Small | |
| Check of | Battery Usage | 1% | 2% | 5% | 6% | 6% |
| Remaining | Ratio Iᵤᵣ | | | | | |
| Battery Level | Remaining | 99% | 98% | 95% | 94% | 94% |
| Iᵣ | Battery | | | | | |
| | Level Ratio Iᵣᵣ | | | | | |

| | Results | Normal | | |
|---|---|---|---|---|
| Check of | Results | Normal | Ab- | Normal |
| Battery Voltage | | | normal | |
| Comprehensive | Results | Normal | Ab- | Normal |
| Determination | | | normal | |

As shown in Table 2, if the first self-diagnosis for the second time is conducted when the battery cell is abnormal, the first current, which is low, is supplied from the battery cell to the body without charging the high-voltage power source by the battery cell. Therefore, the battery usage ratio Iᵤᵣ and the remaining battery level ratio Iᵣᵣ are normal value. In addition, since the first current and the voltage drop of the voltage Vₛ measured by the voltmeter are small, the voltage Vₛ exceeds the predetermined threshold Vₜₕ. Therefore, it is determined that the battery voltage is normal and that the state of the defibrillator is normal.

Based on the results of the self-diagnosis, information indicating that the state of the defibrillator is normal is recorded on the memory, and battery information indicating that the battery cell is normal (specifically, according to Table 2, the battery usage ratio Iᵤᵣ is 2%, and the remaining battery level ratio Iᵣᵣ is 98%) is recorded on the battery memory. Accordingly, even if the battery cell is abnormal, the first self-diagnosis cannot detect an abnormality in the battery voltage, and the information indicating that the state of the defibrillator is normal is recorded since the first current and the voltage drop of the voltage Vₛ are small.

If the second self-diagnosis is conducted, the high-voltage power source is charged by the battery cell, and the second current, which is high, is supplied from the battery cell to the body 10. However, since the intervals for the second self-diagnosis are long (the frequency is low), the fluctuation ΔIᵤ in the battery usage is small, and the battery usage ratio Iᵤᵣ and the remaining battery level Iᵣᵣ are normal value. In addition, since the battery cell is abnormal and has large internal impedance, the voltage drop of the voltage Vₛ measured by the voltmeter is large, and the voltage Vₛ is equal to or less than the predetermined threshold Vₜₕ. Therefore, it is determined that the battery voltage is abnormal and that the state of the defibrillator is abnormal.

Based on the results of the self-diagnosis, information indicating that the state of the defibrillator is abnormal is recorded on the memory, and battery information indicating that the battery cell is abnormal (specifically, according to Table 2, the battery usage ratio Iᵤᵣ is 5%, and the remaining battery level ratio Iᵣᵣ is 95%) is recorded on the battery memory.

Since a large current is applied in the second self-diagnosis, the second self-diagnosis can detect an abnormality in the battery voltage that cannot be detected in the first self-diagnosis.

However, as shown in Table 2, if the first self-diagnosis is reconducted after the information indicating that the defibrillator is abnormal is recorded on the memory in the second self-diagnosis, the memory is overwritten with the information indicating that the state of the defibrillator is normal even though the battery cell is abnormal in fact. The memory is overwritten with the latest results since there is a possibility that the results of the second self-diagnosis may temporarily become abnormal due to an abnormality, for example, in peripheral temperature. However, this makes it difficult to detect an abnormality in the internal impedance of the battery cell.

The defibrillator 1 according to the present embodiment remedies such inconvenience. Table 3 shows results of the self-diagnosis for the defibrillator 1 according to the present embodiment when the battery cell 11A is abnormal (when the internal impedance is large).

TABLE 3

| Number of Self-diagnosis | | 1 | 2 | 3 | 4 | 5 |
|---|---|---|---|---|---|---|
| Type of Self-diagnosis | | First | | Second | First | |
| Internal Impedance of Battery | | Normal | Abnormal | | | |
| Cell | | | | | | |
| High-voltage Power Source | | No | | Yes | No | |
| Is Charged? | | | | | | |
| Voltage Drop | | Small | | Large | Small | |
| Check of | Battery | 1% | 2% | 100% | 100% | 100% |
| Remaining | Usage | | | | | |
| Battery | Ratio Iᵤᵣ | | | | | |
| Level | Remaining | 99% | 98% | 0% | 0% | 0% |
| Iᵣ | Battery | | | | | |
| | Level | | | | | |
| | Ratio Iᵣᵣ | | | | | |

| | Results | Normal | Abnormal | |
|---|---|---|---|---|
| Check of | Results | Normal | Abnormal | Normal |
| Battery Voltage | | | | |

| | | | |
|---|---|---|---|
| Comprehensive | Results | Normal | Abnormal |
| Determination | | | |

As shown in Table 3, if the first self-diagnosis is conducted, the first current, which is low, is supplied from the battery cell 11A to the body 10 without charging the high-voltage power source 105 from the battery cell 11A. Therefore, the battery usage ratio Iᵤᵣ and the remaining battery level ratio Iᵣᵣ are normal value. In addition, since the first current and the voltage drop of the voltage Vₛ measured by the voltmeter 108 are small, the voltage Vₛ exceeds the predetermined threshold Vₜₕ. Therefore, it is determined that the battery voltage is normal and that the state of the defibrillator 1 is normal.

Based on the results of the self-diagnosis, information indicating that the state of the defibrillator 1 is normal is recorded on the memory 104, and battery information indicating that the battery cell 11A is normal is recorded on the battery memory 11B.

If the second self-diagnosis is conducted, the high-voltage power source 105 is charged by the battery cell 11A, and the second current, which is high, is supplied from the battery cell 11A to the body 10. However, since the intervals for the second self-diagnosis are long, the fluctuation ΔIᵤ in the battery usage is small, and the battery usage ratio Iᵤᵣ and the remaining battery level ratio Iᵣᵣ are normal value. Also, since the battery cell 11A is abnormal and has large internal impedance, the voltage drop of the voltage Vₛ measured by the voltmeter 108 is large and is below the predetermined threshold Vₜₕ. Therefore, it is determined that the battery voltage is abnormal and that the state of the defibrillator 1 is abnormal.

In this case, instead of the battery usage ratio Iᵤᵣ and the remaining battery level ratio Iᵣᵣ calculated by the processing circuitry 103, a battery usage ratio Iᵤᵣ and a remaining battery level ratio Iᵣᵣ indicating that the battery cell 11A is abnormal (specifically, according to Table 3, the battery usage ratio Iᵤᵣ is 100%, and the remaining battery level ratio Iᵣᵣ is 0%) are recorded on the battery memory 11B. Therefore, information indicating that the state of the defibrillator 1 is abnormal is recorded on the memory 104, and battery information indicating that the battery cell 11A is abnormal is recorded on the battery memory 11B.

As shown in Table 3, in the self-diagnosis of the defibrillator 1 according to the present embodiment, the battery information indicating that the battery cell 11A is abnormal is recorded on the battery memory 11B in the second self-diagnosis (No. 3 in Table 3). Therefore, if the first self-diagnosis is reconducted thereafter (when the self-diagnosis No. 4 in Table 3 is conducted), it is determined that the remaining battery level Iᵣ is abnormal since the remaining battery level ratio Iᵣᵣ is below the predetermined threshold Iₜₕ during the check of the remaining battery level Iᵣ at the start of the self-diagnosis. As a result, the memory 104 is overwritten with information indicating that the state of the defibrillator 1 is abnormal.

Accordingly, even if the first self-diagnosis, in which a low current (first current) is supplied from the battery, is conducted, the users or the administrators can easily recognize information indicating that the state of the defibrillator 1 is abnormal. The self-diagnosis No. 4 is the first self-diagnosis, and the first current and the fluctuation ΔIᵤ in the battery usage are small. However, since the battery information indicating that the battery cell 11A is abnormal has already been recorded on the battery memory 11B, battery information indicating that the battery cell 11A is abnormal is recorded on the battery memory 11B also in the self-diagnosis No. 4.

In the example above, if a voltage abnormality of the battery cell 11A is detected, the processing circuitry 103 records the battery usage ratio Iᵤᵣ and the remaining battery level ratio Iᵣᵣ indicating that the battery cell 11A is abnormal (specifically, according to Table 3, the battery usage ratio Iᵤᵣ is 100% and the remaining battery level ratio Iᵣᵣ is 0%) on the battery memory 11B, but the present disclosure is not limited thereto. For example, the processing circuitry 103 may record a flag having a specific value (for example, “1”) when the voltage is abnormal on the battery memory 11B. However, by recording a battery usage ratio Iᵤᵣ and/or a remaining battery level ratio Iᵣᵣ indicating an abnormality on the battery memory 11B, even if the battery pack 11 is used in a defibrillator having a different model number, the remaining battery level Iᵣ becomes abnormal, and the abnormality in the battery pack 11 can be recognized. In other words, by recording a battery usage ratio Iᵤᵣ and/or a remaining battery level ratio Iᵣᵣ indicating an abnormality on the battery memory 11B, the abnormality can always be detected as long as the defibrillator is configured to check the remaining battery level Iᵣ recorded in the battery memory 11B.

When the battery pack 11 is attached unsteadily, the internal impedance of the battery pack 11 may be temporarily abnormal even if the battery pack 11 itself is normal. Therefore, until a predetermined period of time has passed since the processing circuitry 103 detects that the battery pack 11 is attached to the defibrillator 1, the first and/or the second self-diagnosis may be conducted, but battery information does not have to be recorded on the battery memory 11B, and the voltage Vₛ does not have to be compared with the predetermined threshold Vₜₕ. Accordingly, even if the connection between the battery pack 11 and the defibrillator 1 is unstable when the battery pack 11 is attached to the defibrillator 1, it can be possible to prevent battery information indicating that the battery cell 11A is abnormal from being erroneously recorded on the battery memory 11B.

During the first and/or the second self-diagnosis, the processing circuitry 103 may transmit the battery information to an external server 110 via the external communication unit 109. Accordingly, the users or the administrators can easily recognize the battery usage ratio Iᵤᵣ and the remaining battery level ratio Iᵣᵣ of the battery cell 11A.

Although an embodiment of the present disclosure has been described above, it goes without saying that the technical scope of the present disclosure should not be limited to the present embodiment. It is to be understood by those skilled in the art that the present embodiment is merely an example and that various modifications can be made within the scope of the invention described in the claims. The technical scope of the present disclosure should be determined based on the scope of the inventions described in the claims or the equivalents thereof.

For example, in the above description, the defibrillator 1 is an automated external defibrillator (AED) that is fully automated, but the present disclosure is not limited thereto. The defibrillator 1 may be, for example, an AED that is semi-automated.

What is claimed is:

1. A defibrillator comprising:
processing circuitry; and
a memory storing a program, wherein,
when executed on the processing circuitry, the program causes the processing circuitry to:
calculate battery usage of a battery configured to supply power to the defibrillator;
record, on a battery memory, information related to the battery usage; and
record, on the battery memory, battery information indicating battery failure in response to voltage of the power supplied from the battery being below a threshold.

2. The defibrillator according to claim 1, wherein,
when executed on the processing circuitry, the program causes the processing circuitry to:
conduct first self-diagnosis in which the voltage is measured with first current supplied from the battery to the defibrillator;
conduct second self-diagnosis in which the voltage is measured with second current, which is higher than the first current, supplied from the battery to the defibrillator; and
record, on the battery memory, the battery information indicating the battery failure in response to the voltage being below the threshold during the second self-diagnosis.

3. The defibrillator according to claim 2, wherein
intervals for the second self-diagnosis are longer than those for the first self-diagnosis.

4. The defibrillator according to claim 2, wherein,
when executed on the processing circuitry, the program further causes the processing circuitry to determine, regardless of the voltage, that the battery is abnormal in a case where the battery information indicating the battery failure is recorded on the battery memory.

5. The defibrillator according to claim 2, wherein,
when executed on the processing circuitry, the program further causes the processing circuitry to record, on the battery memory, battery information indicating that the battery usage exceeds a predetermined value in response to the voltage being below the threshold.

6. The defibrillator according to claim 2, further comprising:
a capacitor configured to be supplied with the power from the battery and to store power for an electric shock, wherein,
when executed on the processing circuitry, the program further causes the processing circuitry to:
cause power not to be supplied from the battery to the capacitor during the first self-diagnosis; and
cause the power for the electric shock to be supplied from the battery to the capacitor during the second self-diagnosis.

7. The defibrillator according to claim 1, wherein,
when executed on the processing circuitry, the program further causes the processing circuitry to cause the battery information to be transmitted to a management server.

8. The defibrillator according to claim 1, wherein,
when executed on the processing circuitry, the program further causes the processing circuitry to:
detect that a battery pack including the battery is attached to the defibrillator; and
causes the battery information not to be recorded on the battery memory until a predetermined period of time has passed since it is detected that the battery pack is attached to the defibrillator.

9. The defibrillator according to claim 1, wherein,
when executed on the processing circuitry, the program further causes the processing circuitry to:
detect that a battery pack including the battery is attached to the defibrillator; and
causes comparison of the voltage with the threshold not to be conducted until a predetermined period of time has passed since it is detected that the battery pack is attached to the defibrillator.

10. The defibrillator according to claim 1, wherein,
when executed on the processing circuitry, the program further causes the processing circuitry to calculate a fluctuation in the battery usage, based on a time integral of current supplied from the battery.
